(19) United States
(12) Patent Application Publication
(10) Pub. No.: US 2025/0269042 A1
(43) Pub. Date: Aug. 28, 2025

(54) MULTIFUNCTIONAL PROTEOLYSIS-TARGETING CHIMERA CONJUGATES AND USES IN TREATING NEUROLOGICAL DISEASES

(71) Applicant: BIOXEL INC., Cambridge, MA (US)

(72) Inventors: Patrick R. GLEASON, Millis, MA (US); Elazer R. EDELMAN, Brookline, MA (US)

(73) Assignee: BIOXEL INC., Cambridge, MA (US)

(21) Appl. No.: 19/062,969

(22) Filed: Feb. 25, 2025

Related U.S. Application Data
(60) Provisional application No. 63/558,575, filed on Feb. 27, 2024.

(51) Int. Cl. A61K 47/55; A61K 47/60

(52) U.S. Cl. CPC A61K 47/55; A61K 47/60

(57) ABSTRACT

The present disclosure provides various multifunctional proteolysis-targeting chimera conjugates and uses thereof for the treatment of neurological diseases or disorders. In some embodiments, the chimera conjugates comprise at least one cellular-targeting moiety, one protein-binding moiety, and one ubiquitin ligase recruitment moiety, wherein these elements are conjugated in a manner that facilitates cellular uptake and targeted degradation of specific proteins within cells.

### CROSS-REFERENCE TO RELATED APPLICATIONS

This application claims priority to and the benefit of U.S. Provisional Application No. 63/558,575, filed on Feb. 27, 2024, which is hereby incorporated by reference.

### SEQUENCE LISTING STATEMENT

A Sequence Listing conforming to the rules of WIPO Standard ST.26 is hereby incorporated by reference. The Sequence Listing has been filed as an electronic document encoded as XML in UTF-8 text. The electronic document, created on Feb. 7, 2024, is entitled “P-635126-USP_ST26.xml” and is 439,673 bytes in size.

### FIELD OF THE INVENTION

The present invention relates to various molecular constructs of multifunctional proteolysis-targeting chimera conjugates having at least one cellular-targeting element, one protein-targeting element, and one ubiquitin ligase recruitment element, wherein these elements are conjugated in a manner that facilitates cellular uptake and targeted degradation of specific proteins within cells. The present invention further relates to uses of the multifunctional proteolysis-targeting chimera conjugates for the treatment of neurological diseases or disorders.

### BACKGROUND OF THE INVENTION

Targeting intracellular proteins implicated in the pathogenesis of a variety of diseases, including the leading causes of mortality and morbidity has been a long-standing challenge in the field of drug discovery and development. Many of these proteins have been termed “undruggable” due to their lack of traditional small-molecule binding sites or the issues related to drug delivery and specificity. This problem is further exacerbated when these target proteins are overexpressed or mutated in diseased cells, contributing to disease progression and therapeutic resistance.

One promising approach to circumvent these challenges is a class of bifunctional molecules capable of recruiting the cellular ubiquitin-proteasome system to selectively degrade target proteins known as Proteolysis-Targeting Chimeras (PROTACs). Traditional PROTACs comprise two distinct moieties: a protein-targeting moiety and an effector moiety that recruits a ubiquitin ligase to the protein of interest for selective degradation. Several of these molecules have shown promise in preclinical pharmacological studies for their ability to degrade proteins that are typically difficult to target with conventional small molecules.

Yet, despite these advances, delivering PROTACs to diseased cells remains a challenge. The non-specific distribution of the PROTAC during circulation in the body can lead to off-target effects and decreased therapeutic efficacy. In addition, many potential target proteins are located inside cells, further complicating the delivery of these therapeutic agents.

The present invention overcomes these issues by incorporating additional functionality to the PROTAC molecule with cellular targeting peptides. These cellular targeting peptides are designed to bind to specific receptors overexpressed on the surface of diseased cells, guiding the delivery of attached therapeutics specifically to the cell of interest. This targeted delivery approach can increase the therapeutic index by enhancing efficacy and reducing systemic toxicity.

### SUMMARY OF THE INVENTION

In one aspect, the present invention provides a chimera conjugate comprising
(a) two or more active moieties,
(b) a central molecule, and
(c) two or more linking groups;
   wherein each of the two or more active moieties is independently linked to the central molecule through a linking group;
   wherein the active moieties comprise a variety of functions that target extracellular elements, intracellular elements, or intracellular processes;
   wherein the active moieties are synergistic; and
   wherein the active moieties have a molecular weight of from 100 Da to 10⁶ Da.

In some embodiments, the chimera conjugate is represented by the structure of Formula (I):

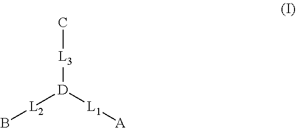

wherein
   A is a cellular targeting moiety;
   B is a protein-binding moiety;
   C is a ubiquitin ligase recruitment moiety;
   D is a central linking moiety; and
   each of L1, L2, and L3 is independently a linking group.

In another aspect, the present invention provides a method for treating a neurological disease or disorder characterized by the aggregation of specific intracellular proteins, comprising administering to a subject in need thereof a therapeutically effective amount of a chimera conjugate described herein. In some embodiments, the intracellular protein is alpha-synuclein, aggregates of alpha-synuclein, or mutants of alpha-synuclein. In some embodiments of the method disclosed herein, the neurological disease or disorder can be, but is not limited to, Parkinson's Disease (PD), Multiple System Atrophy (MSA), Dementia with Lewy Bodies (DLB), Pure Autonomic Failure (PAF), and Neurodegeneration with Brain Iron Accumulation (NBIA).

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present disclosure extends the class of heterobifunctional compounds known as Proteolysis-Targeting Chimeras (PROTACs) to allow a more significant number of active moieties through a more significant number of linkages. This novel therapeutic approach utilizes a multifunctional compound comprising a homing molecule—to selectively bind to receptors of the desired cellular target—conjugated with a molecule constructed to interact with a previously intractable intracellular target and an element(s) that activates the cellular proteolytic pathways. Conjugation of each moiety occurs through a set of linker molecules whose chemistry allows for distinct functionality and the optimization of intracellular target degradation resulting in a therapeutic response. In some embodiments, this provides a potential treatment by modulation of protein populations in various diseases such as Parkinson's Disease (PD), Multiple System Atrophy (MSA), Dementia with Lewy Bodies (DLB), Pure Autonomic Failure (PAF), or Neurodegeneration with Brain Iron Accumulation (NBIA).

In one embodiment, the present disclosure provides novel chimera conjugates designed for targeted intracellular protein degradation. The chimera conjugates are characterized by their unique structures and multifunctionalities that allow them to target specific cellular elements and processes.

In one embodiment, the chimera conjugates disclosed herein have one or more of the following structural components and features:
(1) The conjugates comprises:
   (i) two or more active moieties,
   (ii) a central molecule, and
   (iii) two or more linking groups.
(2) Each active moiety is independently linked to the central molecule through a linking group.
(3) The active moieties are designed to target extracellular elements, intracellular elements, or intracellular processes. In one embodiment, they possess a molecular weight ranging from 100 Da to 10⁶ Da.
(4) The active moieties are synergistic in nature, enhancing the overall efficacy of the chimera conjugates.

In one embodiment, the chimera conjugates disclosed herein comprise
(a) two or more active moieties,
(b) a central molecule, and
(c) two or more linking groups;

wherein each of the two or more active moieties is independently linked to the central molecule through a linking group;

wherein the active moieties comprise a variety of functions that target extracellular elements, intracellular elements, or intracellular processes; and

wherein the active moieties are synergistic.

In some embodiments, the active moieties have a molecular weight of from 100 Da to 10⁶ Da.

In another embodiment, the chimera conjugates disclosed herein comprise
(a) two or more active moieties,
(b) a central molecule, and
(c) two or more linking groups;
   wherein each of the two or more active moieties is independently linked to the central molecule through a linking group;
   wherein the active moieties comprise a variety of functions that target extracellular elements, intracellular elements, and intracellular processes;
   wherein the active moieties are synergistic; and
   wherein the active moieties have a molecular weight of from 100 Da to 10⁶ Da.

In another embodiment, the chimera conjugates disclosed herein comprise
(a) two or more active moieties,
(b) a central molecule, and
(c) two or more linking groups;
   wherein each of the two or more active moieties is independently linked to the central molecule through a linking group;
   wherein the active moieties comprise a variety of functions that target extracellular elements, intracellular elements, or intracellular processes;
   wherein the active moieties are synergistic;
   wherein the active moieties have a molecular weight of from 100 Da to 10⁶ Da; and
   wherein the linking groups have a length of from 1 angstrom to 10 nanometers.

In one embodiment, the chimera conjugates disclosed herein are represented by the structure of Formula (I),

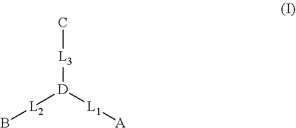

wherein
   A represents a cellular targeting moiety,
   B denotes a protein-binding moiety,
   C denotes an ubiquitin ligase recruitment moiety,
   D is a central linking moiety, and
   L1, L2, and L3 are each independently linking groups.

In some embodiments, the cellular targeting moiety (A) can be a linear peptide or cyclic peptide. In one embodiment, the cellular targeting moiety (A) targets endothelial cells of the blood brain barrier. In one embodiment, the cellular targeting moiety (A) comprises a sequence selected from one of SEQ ID NOs:13-339.

In some embodiments, the protein-binding moiety (B) can be a linear peptide or cyclic peptide. In one embodiment, the protein-binding moiety (B) targets alpha-synuclein, aggregates of alpha-synuclein, or mutants of alpha-synuclein. In one embodiment, the protein-binding moiety (B) comprises a sequence selected from one of SEQ ID NOs:1-12.

In some embodiments, the ubiquitin ligase recruitment moiety (C) is a small molecule with specificity for the CRBN domain of the E3 ligase complex. Examples of such ubiquitin ligase recruitment moiety include, but are not limited to, 2-(2,6-dioxopiperidin-3-yl)-4-hydroxyisoindoline-1,3-dione; 3-(4-amino-1-oxoisoindolin-2-yl)piperidine-2,6-dione; or 4-amino-2-(2,6-dioxopiperidin-3-yl)isoindoline-1,3-dione.

In some embodiments, the ubiquitin ligase recruitment moiety (C) is a small molecule with specificity for the VHL domain of the E3 ligase complex. Example of such ubiquitin ligase recruitment moiety includes, but is not limited to, (2S,4R)-1-((R)-2-amino-3,3-dimethylbutanoyl)-4-methyl-N-(4-(4-methylthiazol-5- yl)benzyl)pyrrolidine-2-carboxamide.

In some embodiments, the ubiquitin ligase recruitment moiety (C) is a small molecule with specificity for the MDM2 domain of the E3 ligase complex. Example of such ubiquitin ligase recruitment moiety includes, but is not limited to, (2R,3S,4R,5S)-N-(4-carbamoyl-2-methoxyphenyl)-3-(3-chloro-2-fluorophenyl)-4-(3-chloro-5-fluorophenyl)-4-ethynyl-5-neopentylpyrrolidine-2-carboxamide.

In some embodiments, the ubiquitin ligase recruitment moiety (C) is a small molecule with specificity for the CIAP domain of the E3 ligase complex. Examples of such ubiquitin ligase recruitment moiety include, but are not limited to, (S)-N-((S)-2-((S)-2-(4-benzoylthiazol-2-yl)pyrrolidin-1-yl)-1-cyclohexyl-2-oxoethyl)-2- (methylamino)propanamide; ((2S,3R)-3-amino-2-hydroxy-4-phenylbutanoyl)leucine; or (S)-2-((S)-1-((S)-2-cyclohexyl-2-((S)-2- (methylamino)propanamido)acetyl)pyrrolidine-2-carboxamido)-3,3-diphenylpropanoic acid.

In some embodiments, the central linking moiety (D) can be an amino acid or its derivative; for example, (D) can be lysine, ornithine, diaminopropionic acid, diaminobutanoic acid, azido lysine, or cysteine.

In some embodiments, the linking groups L1, L2, and L3 are the same or different. In some embodiments, L1, L2, and L3 are each independently a polyethylene glycol (PEG), an amino acid, a hydrocarbon with terminal carboxylic acid and amine groups, or a combination thereof. In some embodiments, the amino acid of L1, L2, and L3 is a canonical or nonstandard amino acid.

In some embodiments, the hydrocarbon with terminal carboxylic acid and amine groups is 5-amino pentanoic acid, 10-amino decanoic acid, or 18-amino octadecanoic acid.

In some embodiments, the linking group L1, L2, and L3 are each independently a polyethylene glycol (PEG). In some embodiments, L1 is PEG2, PEG4, PEG8, or PEG12. In some embodiments, L2 is PEG2, PEG4, PEG8, or PEG12. In some embodiments, L3 is PEG2, PEG4, PEG8, or PEG12.

In some embodiments, L1 comprises a sequence selected from G, GG, GS, GGS, GGGS (SEQ ID NO:340), 2×GGGGS (SEQ ID NO:341), 4×GGGGS (SEQ ID NO:342), or 8×GGGGS (SEQ ID NO:343).

In some embodiments, L2 comprises a sequence selected from G, GG, GS, GGS, GGGS (SEQ ID NO:340), 2×GGGGS (SEQ ID NO:341), 4×GGGGS (SEQ ID NO:342), or 8×GGGGS (SEQ ID NO:343).

In some embodiments, L3 comprises a sequence selected from G, GG, GS, GGS, GGGS (SEQ ID NO:340), 2×GGGGS (SEQ ID NO:341), 4×GGGGS (SEQ ID NO:342), or 8×GGGGS (SEQ ID NO:343).

As used herein, the “cellular targeting moiety” is any peptide capable of targeting either endothelial cells of the blood brain barrier or neuronal cells. The “protein binding peptide” refers to any peptide that is capable of binding to a specific protein with a dissociation constant (K_{d}) less than or equal to 1 μM. The “ubiquitin ligase recruitment moiety” refers to any molecule, small molecule or peptide capable of recruiting an E3 ligase.

In some embodiments, the cellular targeting moiety, the protein binding moiety, and the ubiquitin ligase recruitment moiety are each independently linked to the central linking moiety through a linking group. In some embodiments, the central linking moiety is an amino acid or a derivative thereof.

In some embodiments, the chimera conjugates disclosed herein comprise a cellular targeting moiety, a protein binding moiety, and an ubiquitin ligase recruitment moiety, each independently being connected to a central linking moiety through a linking group, wherein the linking group is a bond, an amino acid, a polyethylene glycol (PEG), or a combination thereof.

In some embodiments, the cellular targeting moiety, the protein binding moiety, and the ubiquitin ligase recruitment moiety are arranged in any order or configuration.

In some embodiments, the chimera conjugates disclosed herein are multifunctional proteolysis-targeting chimera (mPROTAC) conjugates.

The term “multifunctional proteolysis-targeting chimera” and the term “multifunctional proteolysis-targeting chimera conjugate” are used interchangeably herein.

The terms “compound” and “conjugate” are interchangeably used herein, unless the context clearly dictates otherwise.

In some embodiments, the chimera conjugates disclosed herein are capable of degrading a target protein in a cell. In some embodiments, the target protein is alpha-synuclein, aggregates of alpha-synuclein, or mutants of alpha-synuclein.

This disclosure also provides a method for treating a neurological disease or disorder, comprising administering to a subject in need thereof a therapeutically effective amount of a chimera conjugate disclosed herein. In some embodiments of the present method, the neurological disease or disorder is Parkinson's Disease (PD), Multiple System Atrophy (MSA), Dementia with Lewy Bodies (DLB), Pure Autonomic Failure (PAF), or Neurodegeneration with Brain Iron Accumulation (NBIA). In some embodiments, the disease or disorder is resistant to traditional therapeutic regimens. In one embodiment, the method is used to treat Parkinson's disease.

In some embodiments, the chimera conjugates disclosed herein further comprise a fatty diacid, e.g., a C16, to a residue on the peptide of the chimera conjugates to increase plasma half-life.

In some embodiments, the chimera conjugates disclosed herein can be prepared by methods generally known in the art.

In some embodiments, the chimera conjugates disclosed herein are in a pharmaceutically acceptable salt form. Accordingly, it will be appreciated that salt forms of the chimera conjugates are within the scope of the present disclosure.

The term “pharmaceutically acceptable salt” as used herein, in some embodiments, refers to those salts that are safe and effective for pharmaceutical use in mammals and that possess the desired biological activity. Pharmaceutically acceptable salts include salts of acidic or basic groups present in the present chimera conjugates. Pharmaceutically acceptable acid salts include, but are not limited to, hydrochloride, hydrobromide, hydroiodide, nitrate, sulfate, bisulfate, phosphate, acid phosphate, isonicotinate, acetate, lactate, salicylate, citrate, D,L-tartrate, L-tartarate, D-tartarate, pantothenate, bitartrate, ascorbate, succinate, hemisuccinate, maleate, gentisinate, gentisate, fumarate, gluconate, glucaronate, glycolate, saccharate, formate, besylate, benzoate, glutamate, malate, methanesulfonate, ethanesulfonate, benzensulfonate, p-toluenesulfonate, oxalate, tosylate, naphtalen-2-sulfate, or pamoate (i.e., 1,1′-methylene-bis-(2-hydroxy-3-naphthoate)) salts. Certain chimera conjugates disclosed herein can form pharmaceutically acceptable salts with various amino acids. Suitable base salts include, but are not limited to, aluminum, calcium, lithium, magnesium, potassium, sodium, zinc, and diethanolamine salts.

In another aspect, the present disclosure provides a pharmaceutical composition comprising a mPROTAC conjugate or a chimera conjugate disclosed herein, or a pharmaceutically acceptable salt thereof, in combination with one or more pharmaceutically acceptable carriers.

The term “pharmaceutically acceptable carrier” is art-recognized and refers to a pharmaceutically acceptable material, composition, or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The route of administration of the compounds or conjugates or pharmaceutical compositions as described herein may be any of those commonly known in the art. For example, the administration can be by any appropriate mode, including orally, parenterally, intravenously, intramuscularly, intraperitoneally, transdermally, intranasally, or by direct infusion with a catheter. The dosage and frequency of administration will depend on the condition of the patient, concurrent administration of other drugs, and other parameters to be taken into account by the clinician.

A proteolysis-targeting chimera (PROTAC) is classically composed of two key components linked together by a single linking group. The current disclosure dramatically expands the notion of PROTACs by adding multifunctionality (mPROTAC) with cellular targeting moieties. This approach allows for distinct moieties to allow for specific control of each element: cell binding, internalization, target interaction, and target modification.

The mPROTAC conjugate of the present disclosure can be used in treatment for various diseases such as parkinson's disease where a peptide is specifically designed to selectively enter the brain by passing through the blood brain barrier followed by penetration of neuronal cells. This facet of the mPROTAC conjugate facilitates the accumulation of the therapeutic molecule specifically in the brain, reducing systemic exposure and potential off-target effects while also increasing efficacy.

The protein-binding moiety of the mPROTAC conjugate is designed to engage with an intracellular target protein implicated in cellular homeostasis. Once inside the diseased cell, this element of the mPROTAC conjugate binds to the target protein, initiating its degradation via the proteasomal pathway. By utilizing the cell's own ubiquitin-proteasome system, the mPROTAC of the present disclosure triggers the degradation of the target protein, effectively disrupting the intracellular processes essential for the survival and proliferation of diseased cells.

The mPROTAC conjugate of the present disclosure provides a significant advancement in pharmacotherapy, offering a potent, selective, and potentially safer alternative to conventional drug treatments.

The present disclosure presents a unique PROTAC conjugate capable of selective delivery and targeted therapeutic effect on nerve cells. This innovative design offers a promising new approach to treat neurological disease or disorder, combining targeted delivery, protein interaction specificity, and induced proteasomal degradation.

1. A multifunctional proteolysis-targeting chimera conjugate having the structure of Formula (I) 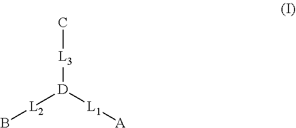
wherein
A is a cellular targeting moiety;
B is a protein-binding moiety;
C is a ubiquitin ligase recruitment moiety;
D is a central linking moiety; and
each of L1, L2, and L3 is independently a linking group.

2. The conjugate of claim 1, wherein the cellular targeting moiety (A) is a linear peptide or a cyclic peptide.

3. The conjugate of claim 1, wherein the cellular targeting moiety (A) targets endothelial cells of the blood brain barrier.

4. The conjugate of claim 3, wherein the cellular targeting moiety (A) comprises a sequence selected from one of SEQ ID NOs:13-339.

5. The conjugate of claim 1, wherein the protein-binding moiety (B) targets alpha-synuclein or mutants of alpha-synuclein.

6. The conjugate of claim 5, wherein the protein-binding moiety (B) comprises a sequence selected from one of SEQ ID NOs:1-12.

7. The conjugate of claim 1, wherein the ubiquitin ligase recruitment moiety (C) is a small molecule with specificity for the CRBN domain of the E3 ligase complex, the VHL domain of the E3 ligase complex, the MDM2 domain of the E3 ligase complex, or the CIAP domain of the E3 ligase complex.

8. The conjugate of claim 7, wherein the ubiquitin ligase recruitment moiety (C) is selected from the group consisting of 2-(2,6-dioxopiperidin-3-yl)-4-hydroxyisoindoline-1,3-dione, 3-(4-amino-1-oxoisoindolin-2-yl)piperidine-2,6-dione, 4-amino-2-(2,6-dioxopiperidin-3-yl)isoindoline-1,3-dione, (2S,4R)-1-((R)-2-amino-3,3-dimethylbutanoyl)-4-methyl-N-(4-(4-ethylthiazol-5-yl)benzyl)pyrrolidine-2-carboxamide, (2R,3S,4R,5S)-N-(4-carbamoyl-2-methoxyphenyl)-3-(3-chloro-2-fluorophenyl)-4-(3-chloro-5-fluorophenyl)-4-ethynyl-5-neopentylpyrrolidine-2-carboxamide, (S)-N-((S)-2-((S)-2-(4-benzoylthiazol-2-yl)pyrrolidin-1-yl)-1-cyclohexyl-2-oxoethyl)-2-(methylamino)propanamide, ((2S,3R)-3-amino-2-hydroxy-4-phenylbutanoyl)leucine, and(S)-2-((S)-1-((S)-2-cyclohexyl-2-((S)-2-(methylamino)propanamido)acetyl)pyrrolidine-2-carboxamido)-3,3-diphenylpropanoic acid.

9.-18. (canceled)

19. The conjugate of claim 1, wherein (D) is an amino acid or a derivative thereof.

20. (canceled)

21. The conjugate of claim 19, wherein (D) is selected from lysine azido lysine or cysteine.

22. The conjugate of claim 1, wherein L1, L2, and L3 are the same or different.

23. The conjugate of claim 1, wherein L1, L2, and L3 are each independently selected from a polyethylene glycol (PEG), an amino acid, a hydrocarbon with terminal carboxylic acid and amine groups, or a combination thereof.

24. The conjugate of claim 23, wherein the amino acid is a canonical or a nonstandard amino acid.

25. The conjugate of claim 23, wherein the hydrocarbon with terminal carboxylic acid and amine groups is 5-amino pentanoic acid, 10-amino decanoic acid, or 18-amino octadecanoic acid.

26. The conjugate of claim 1, wherein L1, L2, and L3 are each independently a polyethylene glycol (PEG).

27. The conjugate of claim 1, wherein L1 is PEG2, PEG4, PEG8, PEG12 or is a sequence selected from G, GG, GS, GGS, GGGS (SEQ ID NO:340), 2×GGGGS (SEQ ID NO:341), 4×GGGGS (SEQ ID NO:342), or 8×GGGGS (SEQ ID NO:343).

28. The conjugate of claim 1, wherein L2 is PEG2, PEG4, PEG8, PEG12, or is a sequence selected from G, GG, GS, GGS, GGGS (SEQ ID NO:340), 2×GGGGS (SEQ ID NO:341), 4×GGGGS (SEQ ID NO:342), or 8×GGGGS (SEQ ID NO:343).

29. The conjugate of claim 1, wherein L3 is PEG2, PEG4, PEG8, PEG12 or is a sequence selected from G, GG, GS, GGS, GGGS (SEQ ID NO:340), 2×GGGGS (SEQ ID NO:341), 4×GGGGS (SEQ ID NO:342), or 8×GGGGS (SEQ ID NO:343).

30. (canceled)

31. (canceled)

32. (canceled)

33. A method for treating a neurological disease or disorder, comprising administering to a subject in need thereof a therapeutically effective amount of the conjugate of claim 1.

34. The method of claim 33, wherein the neurological disease or disorder is Parkinson's Disease (PD), Multiple System Atrophy (MSA), Dementia with Lewy Bodies (DLB), Pure Autonomic Failure (PAF), or Neurodegeneration with Brain Iron Accumulation (NBIA).


SEQUENCE LISTING (343 sequences)

SEQ ID NO: 1 - protein, 10 aa, synthetic construct
  REGION 1..10: note = Description of sequence: potential alpha-synuclein binding sequence
GVLYVGSKTR

SEQ ID NO: 2 - protein, 10 aa, synthetic construct
  REGION 1..10: note = Description of sequence: potential alpha-synuclein binding sequence
  SITE 1: note = D-Arginine
  SITE 2: note = D-Threonine
  SITE 3: note = D-Lysine
  SITE 4: note = D-Serine
  SITE 5: note = D-Glycine
  SITE 6: note = D-Valine
  SITE 7: note = D-Tyrosine
  SITE 8: note = D-Leucine
  SITE 9: note = D-Valine
  SITE 10: note = D-Glycine
RTKSGVYLVG

SEQ ID NO: 3 - protein, 12 aa, synthetic construct
  REGION 1..12: note = Description of sequence: potential alpha-synuclein binding sequence
CDLCRDMLELEE

SEQ ID NO: 4 - protein, 15 aa, synthetic construct
  REGION 1..15: note = Description of sequence: potential alpha-synuclein binding sequence
DPAQKCDRDWMRDMC

SEQ ID NO: 5 - protein, 16 aa, synthetic construct
  REGION 1..16: note = Description of sequence: potential alpha-synuclein binding sequence
AAQKADTDWMRDMCDD

SEQ ID NO: 6 - protein, 15 aa, synthetic construct
  REGION 1..15: note = Description of sequence: potential alpha-synuclein binding sequence
TAELELMLDTQEDCM

SEQ ID NO: 7 - protein, 14 aa, synthetic construct
  REGION 1..14: note = Description of sequence: potential alpha-synuclein binding sequence
AAAATADWMEMMCD

SEQ ID NO: 8 - protein, 16 aa, synthetic construct
  REGION 1..16: note = Description of sequence: potential alpha-synuclein binding sequence
AAAAVFTDPMCRDMCD

SEQ ID NO: 9 - protein, 14 aa, synthetic construct
  REGION 1..14: note = Description of sequence: potential alpha-synuclein binding sequence
LAALCELLDDMQEL

SEQ ID NO: 10 - protein, 14 aa, synthetic construct
  REGION 1..14: note = Description of sequence: potential alpha-synuclein binding sequence
DAQACDRDWMRDMC

SEQ ID NO: 11 - protein, 15 aa, synthetic construct
  REGION 1..15: note = Description of sequence: potential alpha-synuclein binding sequence
TAAAALTMALDWWHD

SEQ ID NO: 12 - protein, 16 aa, synthetic construct
  REGION 1..16: note = Description of sequence: potential alpha-synuclein binding sequence
DPAAVVRQACDCWEDM

SEQ ID NO: 13 - protein, 16 aa, synthetic construct
  REGION 1..16: note = Description of sequence: Blood brain barrier penetrating sequence
RQIKIWFQNRRMKWKK

SEQ ID NO: 14 - protein, 22 aa, synthetic construct
  REGION 1..22: note = Description of sequence: Blood brain barrier penetrating sequence
FVPIFTHSELQKIREKERNKGQ

SEQ ID NO: 15 - protein, 10 aa, synthetic construct
  REGION 1..10: note = Description of sequence: Blood brain barrier penetrating sequence
KQTENLADTY

SEQ ID NO: 16 - protein, 17 aa, synthetic construct
  REGION 1..17: note = Description of sequence: Blood brain barrier penetrating sequence
LLGMIPVAISAINLMKL

SEQ ID NO: 17 - protein, 25 aa, synthetic construct
  REGION 1..25: note = Description of sequence: Blood brain barrier penetrating sequence
GLWDDLKAAAKKVVSSLASAAIEKL

SEQ ID NO: 18 - protein, 18 aa, synthetic construct
  REGION 1..18: note = Description of sequence: Blood brain barrier penetrating sequence
NSGMINSILGIPRVMTEA

SEQ ID NO: 19 - protein, 6 aa, synthetic construct
  REGION 1..6: note = Description of sequence: Blood brain barrier penetrating sequence
NPTNLH

SEQ ID NO: 20 - protein, 19 aa, synthetic construct
  REGION 1..19: note = Description of sequence: Blood brain barrier penetrating sequence
GIVDFAKGVLGKIKNVLGI

SEQ ID NO: 21 - protein, 11 aa, synthetic construct
  REGION 1..11: note = Description of sequence: Blood brain barrier penetrating sequence
QQDYGTGWFDF

SEQ ID NO: 22 - protein, 5 aa, synthetic construct
  REGION 1..5: note = Description of sequence: Blood brain barrier penetrating sequence
FPPWM

SEQ ID NO: 23 - protein, 10 aa, synthetic construct
  REGION 1..10: note = Description of sequence: Blood brain barrier penetrating sequence
AGVWTVWGTI

SEQ ID NO: 24 - protein, 8 aa, synthetic construct
  REGION 1..8: note = Description of sequence: Blood brain barrier penetrating sequence
DPGFSSWG

SEQ ID NO: 25 - protein, 8 aa, synthetic construct
  REGION 1..8: note = Description of sequence: Blood brain barrier penetrating sequence
DPPFSPRL

SEQ ID NO: 26 - protein, 7 aa, synthetic construct
  REGION 1..7: note = Description of sequence: Blood brain barrier penetrating sequence
IYEPEIA

SEQ ID NO: 27 - protein, 13 aa, synthetic construct
  REGION 1..13: note = Description of sequence: Blood brain barrier penetrating sequence
ILPLVGNLLNDLL

SEQ ID NO: 28 - protein, 10 aa, synthetic construct
  REGION 1..10: note = Description of sequence: Blood brain barrier penetrating sequence
KTCENLADDY

SEQ ID NO: 29 - protein, 14 aa, synthetic construct
  REGION 1..14: note = Description of sequence: Blood brain barrier penetrating sequence
MANTQNISIWWWAR

SEQ ID NO: 30 - protein, 22 aa, synthetic construct
  REGION 1..22: note = Description of sequence: Blood brain barrier penetrating sequence
GLWEKVKEKANELVSGIVEGVK

SEQ ID NO: 31 - protein, 15 aa, synthetic construct
  REGION 1..15: note = Description of sequence: Blood brain barrier penetrating sequence
MNRVQFNHHHHHHPD

SEQ ID NO: 32 - protein, 8 aa, synthetic construct
  REGION 1..8: note = Description of sequence: Blood brain barrier penetrating sequence
CEHSHDGA

SEQ ID NO: 33 - protein, 14 aa, synthetic construct
  REGION 1..14: note = Description of sequence: Blood brain barrier penetrating sequence
FLPILINLIHKGLL

SEQ ID NO: 34 - protein, 26 aa, synthetic construct
  REGION 1..26: note = Description of sequence: Blood brain barrier penetrating sequence
MSGCGCASTGTCHCGKDCTCAGCPHK

SEQ ID NO: 35 - protein, 8 aa, synthetic construct
  REGION 1..8: note = Description of sequence: Blood brain barrier penetrating sequence
AGPYSFGL

SEQ ID NO: 36 - protein, 13 aa, synthetic construct
  REGION 1..13: note = Description of sequence: Blood brain barrier penetrating sequence
NFDEIDRSGFGFN

SEQ ID NO: 37 - protein, 5 aa, synthetic construct
  REGION 1..5: note = Description of sequence: Blood brain barrier penetrating sequence
FPPWE

SEQ ID NO: 38 - protein, 21 aa, synthetic construct
  REGION 1..21: note = Description of sequence: Blood brain barrier penetrating sequence
NPLFGIAGEDGPTGPSGIVGQ

SEQ ID NO: 39 - protein, 7 aa, synthetic construct
  REGION 1..7: note = Description of sequence: Blood brain barrier penetrating sequence
KMYDFGL

SEQ ID NO: 40 - protein, 10 aa, synthetic construct
  REGION 1..10: note = Description of sequence: Blood brain barrier penetrating sequence
LSPTQDFMRF

SEQ ID NO: 41 - protein, 7 aa, synthetic construct
  REGION 1..7: note = Description of sequence: Blood brain barrier penetrating sequence
MRTGNAN

SEQ ID NO: 42 - protein, 14 aa, synthetic construct
  REGION 1..14: note = Description of sequence: Blood brain barrier penetrating sequence
SESEVPGMWFGPRL

SEQ ID NO: 43 - protein, 10 aa, synthetic construct
  REGION 1..10: note = Description of sequence: Blood brain barrier penetrating sequence
NPASGFFGMR

SEQ ID NO: 44 - protein, 9 aa, synthetic construct
  REGION 1..9: note = Description of sequence: Blood brain barrier penetrating sequence
GVDSSFVRL

SEQ ID NO: 45 - protein, 9 aa, synthetic construct
  REGION 1..9: note = Description of sequence: Blood brain barrier penetrating sequence
KPNQDFMRF

SEQ ID NO: 46 - protein, 24 aa, synthetic construct
  REGION 1..24: note = Description of sequence: Blood brain barrier penetrating sequence
VIPFVASVAAEMMQHIFCAASRKC

SEQ ID NO: 47 - protein, 27 aa, synthetic construct
  REGION 1..27: note = Description of sequence: Blood brain barrier penetrating sequence
APVSVGGGTVLAKMYPRGNHWAVGHLM

SEQ ID NO: 48 - protein, 14 aa, synthetic construct
  REGION 1..14: note = Description of sequence: Blood brain barrier penetrating sequence
MKATFVLHGWWRTS

SEQ ID NO: 49 - protein, 11 aa, synthetic construct
  REGION 1..11: note = Description of sequence: Blood brain barrier penetrating sequence
EQFEDYGHMRF

SEQ ID NO: 50 - protein, 16 aa, synthetic construct
  REGION 1..16: note = Description of sequence: Blood brain barrier penetrating sequence
QDSGDGWPQQPFVPRL

SEQ ID NO: 51 - protein, 17 aa, synthetic construct
  REGION 1..17: note = Description of sequence: Blood brain barrier penetrating sequence
GIGSALAKAAKLIEGMV

SEQ ID NO: 52 - protein, 12 aa, synthetic construct
  REGION 1..12: note = Description of sequence: Blood brain barrier penetrating sequence
PAASESGFRRDP

SEQ ID NO: 53 - protein, 22 aa, synthetic construct
  REGION 1..22: note = Description of sequence: Blood brain barrier penetrating sequence
FVGAALKVLANVLPPVISWIKQ

SEQ ID NO: 54 - protein, 15 aa, synthetic construct
  REGION 1..15: note = Description of sequence: Blood brain barrier penetrating sequence
GLLGTLGNLLNGLGL

SEQ ID NO: 55 - protein, 20 aa, synthetic construct
  REGION 1..20: note = Description of sequence: Blood brain barrier penetrating sequence
FLSLIPHAINAVSTLVHHSG

SEQ ID NO: 56 - protein, 31 aa, synthetic construct
  REGION 1..31: note = Description of sequence: Blood brain barrier penetrating sequence
GLPTCGETCFGGTCNTPGCTCDSSWPICTHN

SEQ ID NO: 57 - protein, 8 aa, synthetic construct
  REGION 1..8: note = Description of sequence: Blood brain barrier penetrating sequence
QTSFTPRL

SEQ ID NO: 58 - protein, 9 aa, synthetic construct
  REGION 1..9: note = Description of sequence: Blood brain barrier penetrating sequence
AQPSMRLRF

SEQ ID NO: 59 - protein, 15 aa, synthetic construct
  REGION 1..15: note = Description of sequence: Blood brain barrier penetrating sequence
SHTCEICAFAACAGC

SEQ ID NO: 60 - protein, 9 aa, synthetic construct
  REGION 1..9: note = Description of sequence: Blood brain barrier penetrating sequence
LPEQDFMRF

SEQ ID NO: 61 - protein, 24 aa, synthetic construct
  REGION 1..24: note = Description of sequence: Blood brain barrier penetrating sequence
GIKDWIKGAAKKLIKTVASHIANQ

SEQ ID NO: 62 - protein, 24 aa, synthetic construct
  REGION 1..24: note = Description of sequence: Blood brain barrier penetrating sequence
FFPAIFRLVAKVVPSIICSVTKKC

SEQ ID NO: 63 - protein, 9 aa, synthetic construct
  REGION 1..9: note = Description of sequence: Blood brain barrier penetrating sequence
QEYRGWMDF

SEQ ID NO: 64 - protein, 17 aa, synthetic construct
  REGION 1..17: note = Description of sequence: Blood brain barrier penetrating sequence
QQGEGGPYGGLSPLRFS

SEQ ID NO: 65 - protein, 12 aa, synthetic construct
  REGION 1..12: note = Description of sequence: Blood brain barrier penetrating sequence
EAAGLIPFPRVG

SEQ ID NO: 66 - protein, 18 aa, synthetic construct
  REGION 1..18: note = Description of sequence: Blood brain barrier penetrating sequence
YLKGCWTKSYPPKPCFSR

SEQ ID NO: 67 - protein, 16 aa, synthetic construct
  REGION 1..16: note = Description of sequence: Blood brain barrier penetrating sequence
KIMAKPSKFYEQLRGR

SEQ ID NO: 68 - protein, 5 aa, synthetic construct
  REGION 1..5: note = Description of sequence: Blood brain barrier penetrating sequence
MFSPQ

SEQ ID NO: 69 - protein, 11 aa, synthetic construct
  REGION 1..11: note = Description of sequence: Blood brain barrier penetrating sequence
GRGGASNYVRL

SEQ ID NO: 70 - protein, 15 aa, synthetic construct
  REGION 1..15: note = Description of sequence: Blood brain barrier penetrating sequence
SKGKKANKDVELARG

SEQ ID NO: 71 - protein, 14 aa, synthetic construct
  REGION 1..14: note = Description of sequence: Blood brain barrier penetrating sequence
FLFPLITSFLSKVL

SEQ ID NO: 72 - protein, 14 aa, synthetic construct
  REGION 1..14: note = Description of sequence: Blood brain barrier penetrating sequence
MAATFALHGWWRTS

SEQ ID NO: 73 - protein, 8 aa, synthetic construct
  REGION 1..8: note = Description of sequence: Blood brain barrier penetrating sequence
QLNYSPDW

SEQ ID NO: 74 - protein, 17 aa, synthetic construct
  REGION 1..17: note = Description of sequence: Blood brain barrier penetrating sequence
AEVQDKGEFLAEGGGVR

SEQ ID NO: 75 - protein, 9 aa, synthetic construct
  REGION 1..9: note = Description of sequence: Blood brain barrier penetrating sequence
APQPYAFGL

SEQ ID NO: 76 - protein, 10 aa, synthetic construct
  REGION 1..10: note = Description of sequence: Blood brain barrier penetrating sequence
PQLGLPFPRV

SEQ ID NO: 77 - protein, 9 aa, synthetic construct
  REGION 1..9: note = Description of sequence: Blood brain barrier penetrating sequence
APSGFMGMR

SEQ ID NO: 78 - protein, 10 aa, synthetic construct
  REGION 1..10: note = Description of sequence: Blood brain barrier penetrating sequence
DSDSAQNLIG

SEQ ID NO: 79 - protein, 7 aa, synthetic construct
  REGION 1..7: note = Description of sequence: Blood brain barrier penetrating sequence
GGKYMRF

SEQ ID NO: 80 - protein, 9 aa, synthetic construct
  REGION 1..9: note = Description of sequence: Blood brain barrier penetrating sequence
QLTFTSSWG

SEQ ID NO: 81 - protein, 23 aa, synthetic construct
  REGION 1..23: note = Description of sequence: Blood brain barrier penetrating sequence
GLLQTIKEKLKEFAGGVVTGVQS

SEQ ID NO: 82 - protein, 16 aa, synthetic construct
  REGION 1..16: note = Description of sequence: Blood brain barrier penetrating sequence
TDVKESEFIAEGAVGR

SEQ ID NO: 83 - protein, 11 aa, synthetic construct
  REGION 1..11: note = Description of sequence: Blood brain barrier penetrating sequence
QQDYTGAHFDF

SEQ ID NO: 84 - protein, 17 aa, synthetic construct
  REGION 1..17: note = Description of sequence: Blood brain barrier penetrating sequence
EGANSNEAKGMWFGPRL

SEQ ID NO: 85 - protein, 5 aa, synthetic construct
  REGION 1..5: note = Description of sequence: Blood brain barrier penetrating sequence
FPPWL

SEQ ID NO: 86 - protein, 9 aa, synthetic construct
  REGION 1..9: note = Description of sequence: Blood brain barrier penetrating sequence
CYFNNCPVG

SEQ ID NO: 87 - protein, 25 aa, synthetic construct
  REGION 1..25: note = Description of sequence: Blood brain barrier penetrating sequence
YKVDEDLQGAGGIQSRGYFFFRPRN

SEQ ID NO: 88 - protein, 8 aa, synthetic construct
  REGION 1..8: note = Description of sequence: Blood brain barrier penetrating sequence
DPSFNSWG

SEQ ID NO: 89 - protein, 15 aa, synthetic construct
  REGION 1..15: note = Description of sequence: Blood brain barrier penetrating sequence
YAEVPSPAAQAPTAD

SEQ ID NO: 90 - protein, 12 aa, synthetic construct
  REGION 1..12: note = Description of sequence: Blood brain barrier penetrating sequence
GTLGNLPAPFPG

SEQ ID NO: 91 - protein, 9 aa, synthetic construct
  REGION 1..9: note = Description of sequence: Blood brain barrier penetrating sequence
NPQQDFMRF

SEQ ID NO: 92 - protein, 9 aa, synthetic construct
  REGION 1..9: note = Description of sequence: Blood brain barrier penetrating sequence
SDYLQLARG

SEQ ID NO: 93 - protein, 9 aa, synthetic construct
  REGION 1..9: note = Description of sequence: Blood brain barrier penetrating sequence
AWQDLNAGW

SEQ ID NO: 94 - protein, 11 aa, synthetic construct
  REGION 1..11: note = Description of sequence: Blood brain barrier penetrating sequence
PASDSGFLRDP

SEQ ID NO: 95 - protein, 37 aa, synthetic construct
  REGION 1..37: note = Description of sequence: Blood brain barrier penetrating sequence
EDEECAKTDQICPPNAPNYCCSGSCVPHPRLRIFVCA

SEQ ID NO: 96 - protein, 10 aa, synthetic construct
  REGION 1..10: note = Description of sequence: Blood brain barrier penetrating sequence
AAASDNFMRF

SEQ ID NO: 97 - protein, 25 aa, synthetic construct
  REGION 1..25: note = Description of sequence: Blood brain barrier penetrating sequence
GIGAAILSAGKSIIKGLANGLAEHF

SEQ ID NO: 98 - protein, 15 aa, synthetic construct
  REGION 1..15: note = Description of sequence: Blood brain barrier penetrating sequence
GLLGMVGSLLGGLGL

SEQ ID NO: 99 - protein, 11 aa, synthetic construct
  REGION 1..11: note = Description of sequence: Blood brain barrier penetrating sequence
QQDYTGAHMDF

SEQ ID NO: 100 - protein, 17 aa, synthetic construct
  REGION 1..17: note = Description of sequence: Blood brain barrier penetrating sequence
SGDTSSQAKGMWFGPRL

SEQ ID NO: 101 - protein, 23 aa, synthetic construct
  REGION 1..23: note = Description of sequence: Blood brain barrier penetrating sequence
GIGKFLHSAKKFGKAFVGEIMNS

SEQ ID NO: 102 - protein, 8 aa, synthetic construct
  REGION 1..8: note = Description of sequence: Blood brain barrier penetrating sequence
TNRNFLRF

SEQ ID NO: 103 - protein, 24 aa, synthetic construct
  REGION 1..24: note = Description of sequence: Blood brain barrier penetrating sequence
FLPLLAGLAANFLPTIICKISYKC

SEQ ID NO: 104 - protein, 18 aa, synthetic construct
  REGION 1..18: note = Description of sequence: Blood brain barrier penetrating sequence
GFVDFLKKVAGTIANVVT

SEQ ID NO: 105 - protein, 13 aa, synthetic construct
  REGION 1..13: note = Description of sequence: Blood brain barrier penetrating sequence
GLLDILKKVGKVA

SEQ ID NO: 106 - protein, 15 aa, synthetic construct
  REGION 1..15: note = Description of sequence: Blood brain barrier penetrating sequence
DPMPAEDIVDLAYES

SEQ ID NO: 107 - protein, 9 aa, synthetic construct
  REGION 1..9: note = Description of sequence: Blood brain barrier penetrating sequence
NEEVLFGGR

SEQ ID NO: 108 - protein, 8 aa, synthetic construct
  REGION 1..8: note = Description of sequence: Blood brain barrier penetrating sequence
QVNFTPGW

SEQ ID NO: 109 - protein, 13 aa, synthetic construct
  REGION 1..13: note = Description of sequence: Blood brain barrier penetrating sequence
MTRVQFKHHHHPD

SEQ ID NO: 110 - protein, 9 aa, synthetic construct
  REGION 1..9: note = Description of sequence: Blood brain barrier penetrating sequence
YSLRARPRF

SEQ ID NO: 111 - protein, 36 aa, synthetic construct
  REGION 1..36: note = Description of sequence: Blood brain barrier penetrating sequence
CIKNGNGCQPNGSQGNCCSGYCHKQPGWVAGYCRRK

SEQ ID NO: 112 - protein, 15 aa, synthetic construct
  REGION 1..15: note = Description of sequence: Blood brain barrier penetrating sequence
AHRFAAEDFGALDTA

SEQ ID NO: 113 - protein, 26 aa, synthetic construct
  REGION 1..26: note = Description of sequence: Blood brain barrier penetrating sequence
GLLQTIKEKLESLESLAKGIVSGIQA

SEQ ID NO: 114 - protein, 8 aa, synthetic construct
  REGION 1..8: note = Description of sequence: Blood brain barrier penetrating sequence
GGPYSYGL

SEQ ID NO: 115 - protein, 30 aa, synthetic construct
  REGION 1..30: note = Description of sequence: Blood brain barrier penetrating sequence
SFSVNPAVEILQHRYMEKVAQNNRNFLNRV

SEQ ID NO: 116 - protein, 7 aa, synthetic construct
  REGION 1..7: note = Description of sequence: Blood brain barrier penetrating sequence
QIPWFHR

SEQ ID NO: 117 - protein, 18 aa, synthetic construct
  REGION 1..18: note = Description of sequence: Blood brain barrier penetrating sequence
RRWCFRVCYRGFCYRKCR

SEQ ID NO: 118 - protein, 30 aa, synthetic construct
  REGION 1..30: note = Description of sequence: Blood brain barrier penetrating sequence
ARICPRILMKCKKDSDCLAECICEEHGFCG

SEQ ID NO: 119 - protein, 9 aa, synthetic construct
  REGION 1..9: note = Description of sequence: Blood brain barrier penetrating sequence
CLITNCPRG

SEQ ID NO: 120 - protein, 46 aa, synthetic construct
  REGION 1..46: note = Description of sequence: Blood brain barrier penetrating sequence
GIFSKFGGKAIKNLFIKGAKNIGKEVGMDVIRTGIDVAGCKIKGEC

SEQ ID NO: 121 - protein, 10 aa, synthetic construct
  REGION 1..10: note = Description of sequence: Blood brain barrier penetrating sequence
TPASGFFGMR

SEQ ID NO: 122 - protein, 10 aa, synthetic construct
  REGION 1..10: note = Description of sequence: Blood brain barrier penetrating sequence
RLCIPYIIPC

SEQ ID NO: 123 - protein, 19 aa, synthetic construct
  REGION 1..19: note = Description of sequence: Blood brain barrier penetrating sequence
AIDYDEDEDGRPKVHVDAR

SEQ ID NO: 124 - protein, 6 aa, synthetic construct
  REGION 1..6: note = Description of sequence: Blood brain barrier penetrating sequence
GSPMFV

SEQ ID NO: 125 - protein, 8 aa, synthetic construct
  REGION 1..8: note = Description of sequence: Blood brain barrier penetrating sequence
GLLSGLGL

SEQ ID NO: 126 - protein, 41 aa, synthetic construct
  REGION 1..41: note = Description of sequence: Blood brain barrier penetrating sequence
MKNTVKLEQFVALKEKDLQKIKGGESRLPKIRFDFIFPRKK

SEQ ID NO: 127 - protein, 17 aa, synthetic construct
  REGION 1..17: note = Description of sequence: Blood brain barrier penetrating sequence
AVNIPFKVKFRCKAAFC

SEQ ID NO: 128 - protein, 7 aa, synthetic construct
  REGION 1..7: note = Description of sequence: Blood brain barrier penetrating sequence
VISIIPV

SEQ ID NO: 129 - protein, 16 aa, synthetic construct
  REGION 1..16: note = Description of sequence: Blood brain barrier penetrating sequence
FLPIVGKSLSGLSGKL

SEQ ID NO: 130 - protein, 10 aa, synthetic construct
  REGION 1..10: note = Description of sequence: Blood brain barrier penetrating sequence
QDVVHSFLRF

SEQ ID NO: 131 - protein, 10 aa, synthetic construct
  REGION 1..10: note = Description of sequence: Blood brain barrier penetrating sequence
GPSSGFFGTR

SEQ ID NO: 132 - protein, 12 aa, synthetic construct
  REGION 1..12: note = Description of sequence: Blood brain barrier penetrating sequence
GGSSGLISMPRV

SEQ ID NO: 133 - protein, 21 aa, synthetic construct
  REGION 1..21: note = Description of sequence: Blood brain barrier penetrating sequence
NEQSGKSQTVIVGPWGAQVST

SEQ ID NO: 134 - protein, 17 aa, synthetic construct
  REGION 1..17: note = Description of sequence: Blood brain barrier penetrating sequence
GSGGSGEANGMWFGPRL

SEQ ID NO: 135 - protein, 28 aa, synthetic construct
  REGION 1..28: note = Description of sequence: Blood brain barrier penetrating sequence
GLLDTIKGVAKTVAASMLDKLKCKISGC

SEQ ID NO: 136 - protein, 36 aa, synthetic construct
  REGION 1..36: note = Description of sequence: Blood brain barrier penetrating sequence
TPLQPKYPGDGAPVEDLIQFYDDLQQYLNVVTRPRF

SEQ ID NO: 137 - protein, 30 aa, synthetic construct
  REGION 1..30: note = Description of sequence: Blood brain barrier penetrating sequence
GLPVCGETCFGGTCNTPGCICDPWPVCTRN

SEQ ID NO: 138 - protein, 10 aa, synthetic construct
  REGION 1..10: note = Description of sequence: Blood brain barrier penetrating sequence
LMYYTLPRPV

SEQ ID NO: 139 - protein, 11 aa, synthetic construct
  REGION 1..11: note = Description of sequence: Blood brain barrier penetrating sequence
PAPDSSFIRDP

SEQ ID NO: 140 - protein, 11 aa, synthetic construct
  REGION 1..11: note = Description of sequence: Blood brain barrier penetrating sequence
DEGGTQYTPRL

SEQ ID NO: 141 - protein, 10 aa, synthetic construct
  REGION 1..10: note = Description of sequence: Blood brain barrier penetrating sequence
QHWSYGWLPG

SEQ ID NO: 142 - protein, 10 aa, synthetic construct
  REGION 1..10: note = Description of sequence: Blood brain barrier penetrating sequence
APASGFFGMR

SEQ ID NO: 143 - protein, 8 aa, synthetic construct
  REGION 1..8: note = Description of sequence: Blood brain barrier penetrating sequence
GAQFSSWG

SEQ ID NO: 144 - protein, 5 aa, synthetic construct
  REGION 1..5: note = Description of sequence: Blood brain barrier penetrating sequence
DILRG

SEQ ID NO: 145 - protein, 9 aa, synthetic construct
  REGION 1..9: note = Description of sequence: Blood brain barrier penetrating sequence
QGLIPFPRV

SEQ ID NO: 146 - protein, 24 aa, synthetic construct
  REGION 1..24: note = Description of sequence: Blood brain barrier penetrating sequence
GFKDWIKGAAKKLIKTVAANIANQ

SEQ ID NO: 147 - protein, 30 aa, synthetic construct
  REGION 1..30: note = Description of sequence: Blood brain barrier penetrating sequence
GIPCGESCVFIPCISSVVGCSCKSKVCYLD

SEQ ID NO: 148 - protein, 24 aa, synthetic construct
  REGION 1..24: note = Description of sequence: Blood brain barrier penetrating sequence
FLPMLAGLAASMVPKFVCLITKKC

SEQ ID NO: 149 - protein, 18 aa, synthetic construct
  REGION 1..18: note = Description of sequence: Blood brain barrier penetrating sequence
LLGMLPVALSALSALSKL

SEQ ID NO: 150 - protein, 25 aa, synthetic construct
  REGION 1..25: note = Description of sequence: Blood brain barrier penetrating sequence
GLWSKIKEAAKTAGLMAMGFVNDMV

SEQ ID NO: 151 - protein, 44 aa, synthetic construct
  REGION 1..44: note = Description of sequence: Blood brain barrier penetrating sequence
GGKCTVDWGGQGGGRRLPSPLFCCYKPTRICYLNQETCETETCP

SEQ ID NO: 152 - protein, 11 aa, synthetic construct
  REGION 1..11: note = Description of sequence: Blood brain barrier penetrating sequence
GASGLIAFPRV

SEQ ID NO: 153 - protein, 6 aa, synthetic construct
  REGION 1..6: note = Description of sequence: Blood brain barrier penetrating sequence
FVPIWM

SEQ ID NO: 154 - protein, 23 aa, synthetic construct
  REGION 1..23: note = Description of sequence: Blood brain barrier penetrating sequence
FLPLVRGAAKLIPSVVCAISKRC

SEQ ID NO: 155 - protein, 29 aa, synthetic construct
  REGION 1..29: note = Description of sequence: Blood brain barrier penetrating sequence
GIPTCGETCTLGTCNTPGCTCSWPICTKN

SEQ ID NO: 156 - protein, 8 aa, synthetic construct
  REGION 1..8: note = Description of sequence: Blood brain barrier penetrating sequence
SPAFNSWG

SEQ ID NO: 157 - protein, 8 aa, synthetic construct
  REGION 1..8: note = Description of sequence: Blood brain barrier penetrating sequence
PDMYAFGL

SEQ ID NO: 158 - protein, 9 aa, synthetic construct
  REGION 1..9: note = Description of sequence: Blood brain barrier penetrating sequence
AAGQDFMRF

SEQ ID NO: 159 - protein, 8 aa, synthetic construct
  REGION 1..8: note = Description of sequence: Blood brain barrier penetrating sequence
GLVSNLGI

SEQ ID NO: 160 - protein, 9 aa, synthetic construct
  REGION 1..9: note = Description of sequence: Blood brain barrier penetrating sequence
CFVRNCPTG

SEQ ID NO: 161 - protein, 9 aa, synthetic construct
  REGION 1..9: note = Description of sequence: Blood brain barrier penetrating sequence
GPETAFLRL

SEQ ID NO: 162 - protein, 14 aa, synthetic construct
  REGION 1..14: note = Description of sequence: Blood brain barrier penetrating sequence
GGGGTSGLFAFPRV

SEQ ID NO: 163 - protein, 9 aa, synthetic construct
  REGION 1..9: note = Description of sequence: Blood brain barrier penetrating sequence
AGLFAQPRL

SEQ ID NO: 164 - protein, 9 aa, synthetic construct
  REGION 1..9: note = Description of sequence: Blood brain barrier penetrating sequence
YSQVSRPRF

SEQ ID NO: 165 - protein, 12 aa, synthetic construct
  REGION 1..12: note = Description of sequence: Blood brain barrier penetrating sequence
GAVSGLLTNLGL

SEQ ID NO: 166 - protein, 17 aa, synthetic construct
  REGION 1..17: note = Description of sequence: Blood brain barrier penetrating sequence
FGETSGETKGMWFGPRL

SEQ ID NO: 167 - protein, 5 aa, synthetic construct
  REGION 1..5: note = Description of sequence: Blood brain barrier penetrating sequence
ACSAG

SEQ ID NO: 168 - protein, 9 aa, synthetic construct
  REGION 1..9: note = Description of sequence: Blood brain barrier penetrating sequence
LPDSSFLRL

SEQ ID NO: 169 - protein, 38 aa, synthetic construct
  REGION 1..38: note = Description of sequence: Blood brain barrier penetrating sequence
VAKCTEESGGKYFVFCCYKPTRICYMNEQKCESTCIGK

SEQ ID NO: 170 - protein, 10 aa, synthetic construct
  REGION 1..10: note = Description of sequence: Blood brain barrier penetrating sequence
QSDDYGHMRF

SEQ ID NO: 171 - protein, 8 aa, synthetic construct
  REGION 1..8: note = Description of sequence: Blood brain barrier penetrating sequence
QLNFSPNW

SEQ ID NO: 172 - protein, 21 aa, synthetic construct
  REGION 1..21: note = Description of sequence: Blood brain barrier penetrating sequence
GSKKPVPIIYCNRRTGKCQRM

SEQ ID NO: 173 - protein, 12 aa, synthetic construct
  REGION 1..12: note = Description of sequence: Blood brain barrier penetrating sequence
GAVPAAQFSPRL

SEQ ID NO: 174 - protein, 36 aa, synthetic construct
  REGION 1..36: note = Description of sequence: Blood brain barrier penetrating sequence
WNPFKELERAGQRVRDAIISAGPAVATVAQATALAK

SEQ ID NO: 175 - protein, 19 aa, synthetic construct
  REGION 1..19: note = Description of sequence: Blood brain barrier penetrating sequence
ATDYDEEEDDRVKVRLDAR

SEQ ID NO: 176 - protein, 8 aa, synthetic construct
  REGION 1..8: note = Description of sequence: Blood brain barrier penetrating sequence
SRPYSFGL

SEQ ID NO: 177 - protein, 9 aa, synthetic construct
  REGION 1..9: note = Description of sequence: Blood brain barrier penetrating sequence
NQEGLFHGR

SEQ ID NO: 178 - protein, 29 aa, synthetic construct
  REGION 1..29: note = Description of sequence: Blood brain barrier penetrating sequence
GWTLNSAGYLLGPHAVDNHRSFNDKHGFT

SEQ ID NO: 179 - protein, 17 aa, synthetic construct
  REGION 1..17: note = Description of sequence: Blood brain barrier penetrating sequence
DGDMSGEGKGMWFGPRL

SEQ ID NO: 180 - protein, 8 aa, synthetic construct
  REGION 1..8: note = Description of sequence: Blood brain barrier penetrating sequence
ARGYDFGL

SEQ ID NO: 181 - protein, 13 aa, synthetic construct
  REGION 1..13: note = Description of sequence: Blood brain barrier penetrating sequence
NEEEKVKWQPDVP

SEQ ID NO: 182 - protein, 9 aa, synthetic construct
  REGION 1..9: note = Description of sequence: Blood brain barrier penetrating sequence
ATGQYAFGL

SEQ ID NO: 183 - protein, 20 aa, synthetic construct
  REGION 1..20: note = Description of sequence: Blood brain barrier penetrating sequence
GYLDYDEVDDNRAKLPLDAR

SEQ ID NO: 184 - protein, 9 aa, synthetic construct
  REGION 1..9: note = Description of sequence: Blood brain barrier penetrating sequence
LFFKGEKKL

SEQ ID NO: 185 - protein, 19 aa, synthetic construct
  REGION 1..19: note = Description of sequence: Blood brain barrier penetrating sequence
CPCGRRRCCVRGLNVYCCF

SEQ ID NO: 186 - protein, 21 aa, synthetic construct
  REGION 1..21: note = Description of sequence: Blood brain barrier penetrating sequence
FLISIPYSASIGGTATLTGTA

SEQ ID NO: 187 - protein, 16 aa, synthetic construct
  REGION 1..16: note = Description of sequence: Blood brain barrier penetrating sequence
MNSVQFKNHHHHHHPD

SEQ ID NO: 188 - protein, 7 aa, synthetic construct
  REGION 1..7: note = Description of sequence: Blood brain barrier penetrating sequence
AGQNTES

SEQ ID NO: 189 - protein, 18 aa, synthetic construct
  REGION 1..18: note = Description of sequence: Blood brain barrier penetrating sequence
SASGGAGESSGMWFGPRL

SEQ ID NO: 190 - protein, 21 aa, synthetic construct
  REGION 1..21: note = Description of sequence: Blood brain barrier penetrating sequence
MLISGTAFLRLRTNRKAFPTP

SEQ ID NO: 191 - protein, 9 aa, synthetic construct
  REGION 1..9: note = Description of sequence: Blood brain barrier penetrating sequence
APTAFYGVR

SEQ ID NO: 192 - protein, 9 aa, synthetic construct
  REGION 1..9: note = Description of sequence: Blood brain barrier penetrating sequence
NIPQLTPTP

SEQ ID NO: 193 - protein, 6 aa, synthetic construct
  REGION 1..6: note = Description of sequence: Blood brain barrier penetrating sequence
HSPGGA

SEQ ID NO: 194 - protein, 7 aa, synthetic construct
  REGION 1..7: note = Description of sequence: Blood brain barrier penetrating sequence
QPWIPFV

SEQ ID NO: 195 - protein, 17 aa, synthetic construct
  REGION 1..17: note = Description of sequence: Blood brain barrier penetrating sequence
GGETGNDAKAMWFGPRL

SEQ ID NO: 196 - protein, 7 aa, synthetic construct
  REGION 1..7: note = Description of sequence: Blood brain barrier penetrating sequence
DPYAFGL

SEQ ID NO: 197 - protein, 15 aa, synthetic construct
  REGION 1..15: note = Description of sequence: Blood brain barrier penetrating sequence
ITKDFDALMKYIKRI

SEQ ID NO: 198 - protein, 10 aa, synthetic construct
  REGION 1..10: note = Description of sequence: Blood brain barrier penetrating sequence
APLMGFQGVR

SEQ ID NO: 199 - protein, 8 aa, synthetic construct
  REGION 1..8: note = Description of sequence: Blood brain barrier penetrating sequence
SPPFAPRL

SEQ ID NO: 200 - protein, 22 aa, synthetic construct
  REGION 1..22: note = Description of sequence: Blood brain barrier penetrating sequence
FVPFFTQSDIQKMQEKERNKGQ

SEQ ID NO: 201 - protein, 25 aa, synthetic construct
  REGION 1..25: note = Description of sequence: Blood brain barrier penetrating sequence
GLLSVLGSVAKHVLPHVVPVIAEHL

SEQ ID NO: 202 - protein, 8 aa, synthetic construct
  REGION 1..8: note = Description of sequence: Blood brain barrier penetrating sequence
QLTFSPDW

SEQ ID NO: 203 - protein, 15 aa, synthetic construct
  REGION 1..15: note = Description of sequence: Blood brain barrier penetrating sequence
VDKPDYRPRPRPPNM

SEQ ID NO: 204 - protein, 11 aa, synthetic construct
  REGION 1..11: note = Description of sequence: Blood brain barrier penetrating sequence
FLPPCAYKGTC

SEQ ID NO: 205 - protein, 10 aa, synthetic construct
  REGION 1..10: note = Description of sequence: Blood brain barrier penetrating sequence
GLLSSLGLNL

SEQ ID NO: 206 - protein, 8 aa, synthetic construct
  REGION 1..8: note = Description of sequence: Blood brain barrier penetrating sequence
GANDFMRF

SEQ ID NO: 207 - protein, 19 aa, synthetic construct
  REGION 1..19: note = Description of sequence: Blood brain barrier penetrating sequence
MGIFSIFVISTVHYQPNKK

SEQ ID NO: 208 - protein, 16 aa, synthetic construct
  REGION 1..16: note = Description of sequence: Blood brain barrier penetrating sequence
TETTEGDFIAEGGGVR

SEQ ID NO: 209 - protein, 13 aa, synthetic construct
  REGION 1..13: note = Description of sequence: Blood brain barrier penetrating sequence
QDKPFWDPPIYPV

SEQ ID NO: 210 - protein, 36 aa, synthetic construct
  REGION 1..36: note = Description of sequence: Blood brain barrier penetrating sequence
ISINQDLKAITDMLLTEQIRERQRYLADLRQRLLEK

SEQ ID NO: 211 - protein, 23 aa, synthetic construct
  REGION 1..23: note = Description of sequence: Blood brain barrier penetrating sequence
ENFSGGCIPGYMRTADGRCKPTY

SEQ ID NO: 212 - protein, 11 aa, synthetic construct
  REGION 1..11: note = Description of sequence: Blood brain barrier penetrating sequence
EAAGLLAFPRV

SEQ ID NO: 213 - protein, 9 aa, synthetic construct
  REGION 1..9: note = Description of sequence: Blood brain barrier penetrating sequence
CYFRNCPIG

SEQ ID NO: 214 - protein, 23 aa, synthetic construct
  REGION 1..23: note = Description of sequence: Blood brain barrier penetrating sequence
SENTGAIGKVFPRGNHWAVGHLM

SEQ ID NO: 215 - protein, 7 aa, synthetic construct
  REGION 1..7: note = Description of sequence: Blood brain barrier penetrating sequence
WKEMSVW

SEQ ID NO: 216 - protein, 9 aa, synthetic construct
  REGION 1..9: note = Description of sequence: Blood brain barrier penetrating sequence
SDYLQLART

SEQ ID NO: 217 - protein, 8 aa, synthetic construct
  REGION 1..8: note = Description of sequence: Blood brain barrier penetrating sequence
LVPFRPRL

SEQ ID NO: 218 - protein, 24 aa, synthetic construct
  REGION 1..24: note = Description of sequence: Blood brain barrier penetrating sequence
FLPIIASVAANVFSKIFCAISKKC

SEQ ID NO: 219 - protein, 9 aa, synthetic construct
  REGION 1..9: note = Description of sequence: Blood brain barrier penetrating sequence
GPDSSFLRL

SEQ ID NO: 220 - protein, 8 aa, synthetic construct
  REGION 1..8: note = Description of sequence: Blood brain barrier penetrating sequence
FDAFTTGF

SEQ ID NO: 221 - protein, 30 aa, synthetic construct
  REGION 1..30: note = Description of sequence: Blood brain barrier penetrating sequence
ERGCPRILKQCKQDSDCPGECICMAHGFCG

SEQ ID NO: 222 - protein, 39 aa, synthetic construct
  REGION 1..39: note = Description of sequence: Blood brain barrier penetrating sequence
QLNFSTGWGRRYADPNADPMAFLTKLIQIEARKLSGCSN

SEQ ID NO: 223 - protein, 30 aa, synthetic construct
  REGION 1..30: note = Description of sequence: Blood brain barrier penetrating sequence
GTPCGESCVYIPCISGVIGCSCTDKVCYLN

SEQ ID NO: 224 - protein, 12 aa, synthetic construct
  REGION 1..12: note = Description of sequence: Blood brain barrier penetrating sequence
GLVPNLLNNLGL

SEQ ID NO: 225 - protein, 22 aa, synthetic construct
  REGION 1..22: note = Description of sequence: Blood brain barrier penetrating sequence
GLGSLLGSLFKFIPKLLPSIQQ

SEQ ID NO: 226 - protein, 30 aa, synthetic construct
  REGION 1..30: note = Description of sequence: Blood brain barrier penetrating sequence
AYPGNGVHCGKYSCTVDKQTAIGNIGNNAA

SEQ ID NO: 227 - protein, 8 aa, synthetic construct
  REGION 1..8: note = Description of sequence: Blood brain barrier penetrating sequence
DEVKIVLD

SEQ ID NO: 228 - protein, 10 aa, synthetic construct
  REGION 1..10: note = Description of sequence: Blood brain barrier penetrating sequence
SPPSQDFMRF

SEQ ID NO: 229 - protein, 11 aa, synthetic construct
  REGION 1..11: note = Description of sequence: Blood brain barrier penetrating sequence
SNRSPSLRLRF

SEQ ID NO: 230 - protein, 19 aa, synthetic construct
  REGION 1..19: note = Description of sequence: Blood brain barrier penetrating sequence
ADGSDPAGGEFLEAGGGVR

SEQ ID NO: 231 - protein, 41 aa, synthetic construct
  REGION 1..41: note = Description of sequence: Blood brain barrier penetrating sequence
MKNTVKLEQFVALKEKDLQKIKGGEMRISRIILDFLFLRKK

SEQ ID NO: 232 - protein, 9 aa, synthetic construct
  REGION 1..9: note = Description of sequence: Blood brain barrier penetrating sequence
SGKPTFIRF

SEQ ID NO: 233 - protein, 13 aa, synthetic construct
  REGION 1..13: note = Description of sequence: Blood brain barrier penetrating sequence
GIAEFLNYIKSKA

SEQ ID NO: 234 - protein, 10 aa, synthetic construct
  REGION 1..10: note = Description of sequence: Blood brain barrier penetrating sequence
QLTFTPNWGT

SEQ ID NO: 235 - protein, 10 aa, synthetic construct
  REGION 1..10: note = Description of sequence: Blood brain barrier penetrating sequence
QLTFSSGWGN

SEQ ID NO: 236 - protein, 19 aa, synthetic construct
  REGION 1..19: note = Description of sequence: Blood brain barrier penetrating sequence
KGAAKGLLEVASCKLSKSC

SEQ ID NO: 237 - protein, 12 aa, synthetic construct
  REGION 1..12: note = Description of sequence: Blood brain barrier penetrating sequence
MLADVFEKIMGD

SEQ ID NO: 238 - protein, 9 aa, synthetic construct
  REGION 1..9: note = Description of sequence: Blood brain barrier penetrating sequence
CYIQSCPIG

SEQ ID NO: 239 - protein, 19 aa, synthetic construct
  REGION 1..19: note = Description of sequence: Blood brain barrier penetrating sequence
GPVGLLSSPGSLPPVGGAP

SEQ ID NO: 240 - protein, 8 aa, synthetic construct
  REGION 1..8: note = Description of sequence: Blood brain barrier penetrating sequence
EPPPWVPV

SEQ ID NO: 241 - protein, 29 aa, synthetic construct
  REGION 1..29: note = Description of sequence: Blood brain barrier penetrating sequence
GLWSKIKDAGKAVLKAAGKAALGAVTDAV

SEQ ID NO: 242 - protein, 15 aa, synthetic construct
  REGION 1..15: note = Description of sequence: Blood brain barrier penetrating sequence
NSVVLGKKQRFHSWG

SEQ ID NO: 243 - protein, 9 aa, synthetic construct
  REGION 1..9: note = Description of sequence: Blood brain barrier penetrating sequence
QGLISFPRV

SEQ ID NO: 244 - protein, 10 aa, synthetic construct
  REGION 1..10: note = Description of sequence: Blood brain barrier penetrating sequence
SPASGFFGMR

SEQ ID NO: 245 - protein, 11 aa, synthetic construct
  REGION 1..11: note = Description of sequence: Blood brain barrier penetrating sequence
PAPESGFIRDP

SEQ ID NO: 246 - protein, 9 aa, synthetic construct
  REGION 1..9: note = Description of sequence: Blood brain barrier penetrating sequence
NQEGLFGGR

SEQ ID NO: 247 - protein, 11 aa, synthetic construct
  REGION 1..11: note = Description of sequence: Blood brain barrier penetrating sequence
DGRTPALRLRF

SEQ ID NO: 248 - protein, 11 aa, synthetic construct
  REGION 1..11: note = Description of sequence: Blood brain barrier penetrating sequence
GSSGLISFPRT

SEQ ID NO: 249 - protein, 7 aa, synthetic construct
  REGION 1..7: note = Description of sequence: Blood brain barrier penetrating sequence
VPPIGWF

SEQ ID NO: 250 - protein, 12 aa, synthetic construct
  REGION 1..12: note = Description of sequence: Blood brain barrier penetrating sequence
GGSSGLIAFPRL

SEQ ID NO: 251 - protein, 14 aa, synthetic construct
  REGION 1..14: note = Description of sequence: Blood brain barrier penetrating sequence
AELPQGLWVRPRLG

SEQ ID NO: 252 - protein, 17 aa, synthetic construct
  REGION 1..17: note = Description of sequence: Blood brain barrier penetrating sequence
GFADLFGKAVDFIKSRV

SEQ ID NO: 253 - protein, 22 aa, synthetic construct
  REGION 1..22: note = Description of sequence: Blood brain barrier penetrating sequence
QRFIHPTYRPPPQPRRPVIMRA

SEQ ID NO: 254 - protein, 8 aa, synthetic construct
  REGION 1..8: note = Description of sequence: Blood brain barrier penetrating sequence
AGPYAFGL

SEQ ID NO: 255 - protein, 10 aa, synthetic construct
  REGION 1..10: note = Description of sequence: Blood brain barrier penetrating sequence
DRTPALRLRF

SEQ ID NO: 256 - protein, 26 aa, synthetic construct
  REGION 1..26: note = Description of sequence: Blood brain barrier penetrating sequence
MSNFHNEHVMQFYRNNLKTKGVFGRQ

SEQ ID NO: 257 - protein, 24 aa, synthetic construct
  REGION 1..24: note = Description of sequence: Blood brain barrier penetrating sequence
FMGGLIKAATKIVPAAYCAITKKC

SEQ ID NO: 258 - protein, 13 aa, synthetic construct
  REGION 1..13: note = Description of sequence: Blood brain barrier penetrating sequence
DAQEKRQPWLPFV

SEQ ID NO: 259 - protein, 12 aa, synthetic construct
  REGION 1..12: note = Description of sequence: Blood brain barrier penetrating sequence
RICTAIPLPMCL

SEQ ID NO: 260 - protein, 19 aa, synthetic construct
  REGION 1..19: note = Description of sequence: Blood brain barrier penetrating sequence
GILDFAKTVVGGIRNALGI

SEQ ID NO: 261 - protein, 14 aa, synthetic construct
  REGION 1..14: note = Description of sequence: Blood brain barrier penetrating sequence
EDTGTFEEGHGGVR

SEQ ID NO: 262 - protein, 13 aa, synthetic construct
  REGION 1..13: note = Description of sequence: Blood brain barrier penetrating sequence
SEEEKRQPWLPFG

SEQ ID NO: 263 - protein, 17 aa, synthetic construct
  REGION 1..17: note = Description of sequence: Blood brain barrier penetrating sequence
FLPLLAASFACTVTKKC

SEQ ID NO: 264 - protein, 10 aa, synthetic construct
  REGION 1..10: note = Description of sequence: Blood brain barrier penetrating sequence
LRQSQFVGSR

SEQ ID NO: 265 - protein, 17 aa, synthetic construct
  REGION 1..17: note = Description of sequence: Blood brain barrier penetrating sequence
KWCFRVCYRGICYRRCR

SEQ ID NO: 266 - protein, 10 aa, synthetic construct
  REGION 1..10: note = Description of sequence: Blood brain barrier penetrating sequence
QHWSDYFKPG

SEQ ID NO: 267 - protein, 30 aa, synthetic construct
  REGION 1..30: note = Description of sequence: Blood brain barrier penetrating sequence
GLPVCGESCFGGTCNTPGCACDPWPVCTRD

SEQ ID NO: 268 - protein, 11 aa, synthetic construct
  REGION 1..11: note = Description of sequence: Blood brain barrier penetrating sequence
QTPQWATGHFM

SEQ ID NO: 269 - protein, 6 aa, synthetic construct
  REGION 1..6: note = Description of sequence: Blood brain barrier penetrating sequence
PGLGFY

SEQ ID NO: 270 - protein, 46 aa, synthetic construct
  REGION 1..46: note = Description of sequence: Blood brain barrier penetrating sequence
MNCLKICGFFFALIAALATAEAGTQVIHAGGHTLIQTDRSQYIRKN

SEQ ID NO: 271 - protein, 21 aa, synthetic construct
  REGION 1..21: note = Description of sequence: Blood brain barrier penetrating sequence
QPSYDYDEEEDDRAKLRLDAR

SEQ ID NO: 272 - protein, 8 aa, synthetic construct
  REGION 1..8: note = Description of sequence: Blood brain barrier penetrating sequence
QSSFHSWG

SEQ ID NO: 273 - protein, 9 aa, synthetic construct
  REGION 1..9: note = Description of sequence: Blood brain barrier penetrating sequence
SVQDNFIRF

SEQ ID NO: 274 - protein, 9 aa, synthetic construct
  REGION 1..9: note = Description of sequence: Blood brain barrier penetrating sequence
ADYLQLARA

SEQ ID NO: 275 - protein, 18 aa, synthetic construct
  REGION 1..18: note = Description of sequence: Blood brain barrier penetrating sequence
EYDDMYTEKRPKVYAFGL

SEQ ID NO: 276 - protein, 11 aa, synthetic construct
  REGION 1..11: note = Description of sequence: Blood brain barrier penetrating sequence
GSTGLIPFGRT

SEQ ID NO: 277 - protein, 8 aa, synthetic construct
  REGION 1..8: note = Description of sequence: Blood brain barrier penetrating sequence
SDPNFLRF

SEQ ID NO: 278 - protein, 24 aa, synthetic construct
  REGION 1..24: note = Description of sequence: Blood brain barrier penetrating sequence
FLPFIARLAAKVFPSIICSVTKKC

SEQ ID NO: 279 - protein, 15 aa, synthetic construct
  REGION 1..15: note = Description of sequence: Blood brain barrier penetrating sequence
GLFDAIGNLLGGLGL

SEQ ID NO: 280 - protein, 30 aa, synthetic construct
  REGION 1..30: note = Description of sequence: Blood brain barrier penetrating sequence
GLAGAISSVLDKLKQSQLIKNYAKKLGYPR

SEQ ID NO: 281 - protein, 8 aa, synthetic construct
  REGION 1..8: note = Description of sequence: Blood brain barrier penetrating sequence
APALRLRF

SEQ ID NO: 282 - protein, 10 aa, synthetic construct
  REGION 1..10: note = Description of sequence: Blood brain barrier penetrating sequence
AYSNLNYLRF

SEQ ID NO: 283 - protein, 11 aa, synthetic construct
  REGION 1..11: note = Description of sequence: Blood brain barrier penetrating sequence
FCTMIPIPRCY

SEQ ID NO: 284 - protein, 11 aa, synthetic construct
  REGION 1..11: note = Description of sequence: Blood brain barrier penetrating sequence
QTLLQELPIPP

SEQ ID NO: 285 - protein, 9 aa, synthetic construct
  REGION 1..9: note = Description of sequence: Blood brain barrier penetrating sequence
WAGGDASGE

SEQ ID NO: 286 - protein, 28 aa, synthetic construct
  REGION 1..28: note = Description of sequence: Blood brain barrier penetrating sequence
RSCPRIWMECTRDSDCMAKCICVAGHCG

SEQ ID NO: 287 - protein, 22 aa, synthetic construct
  REGION 1..22: note = Description of sequence: Blood brain barrier penetrating sequence
MLHHMTSRANLLLLRRGGSQRS

SEQ ID NO: 288 - protein, 11 aa, synthetic construct
  REGION 1..11: note = Description of sequence: Blood brain barrier penetrating sequence
PAPETNYLRDP

SEQ ID NO: 289 - protein, 18 aa, synthetic construct
  REGION 1..18: note = Description of sequence: Blood brain barrier penetrating sequence
NSELINAILGSPTLMGEV

SEQ ID NO: 290 - protein, 10 aa, synthetic construct
  REGION 1..10: note = Description of sequence: Blood brain barrier penetrating sequence
QDVDHVFLRF

SEQ ID NO: 291 - protein, 11 aa, synthetic construct
  REGION 1..11: note = Description of sequence: Blood brain barrier penetrating sequence
QTFHYSQGWTN

SEQ ID NO: 292 - protein, 13 aa, synthetic construct
  REGION 1..13: note = Description of sequence: Blood brain barrier penetrating sequence
GFKNVALSTARGF

SEQ ID NO: 293 - protein, 7 aa, synthetic construct
  REGION 1..7: note = Description of sequence: Blood brain barrier penetrating sequence
GDPFLRF

SEQ ID NO: 294 - protein, 43 aa, synthetic construct
  REGION 1..43: note = Description of sequence: Blood brain barrier penetrating sequence
KYYGNGVSCNKKGCSVDWGKAIGIIGNNSAANLATGGAAGWSK

SEQ ID NO: 295 - protein, 10 aa, synthetic construct
  REGION 1..10: note = Description of sequence: Blood brain barrier penetrating sequence
IIIQYEGHKH

SEQ ID NO: 296 - protein, 7 aa, synthetic construct
  REGION 1..7: note = Description of sequence: Blood brain barrier penetrating sequence
DQVKIVL

SEQ ID NO: 297 - protein, 12 aa, synthetic construct
  REGION 1..12: note = Description of sequence: Blood brain barrier penetrating sequence
FLPIASLLGKYL

SEQ ID NO: 298 - protein, 39 aa, synthetic construct
  REGION 1..39: note = Description of sequence: Blood brain barrier penetrating sequence
GRKSDCFRKSGFCAFLKCPSLTLISGKCSRFYLCCKRIR

SEQ ID NO: 299 - protein, 9 aa, synthetic construct
  REGION 1..9: note = Description of sequence: Blood brain barrier penetrating sequence
PFCNAFTGC

SEQ ID NO: 300 - protein, 18 aa, synthetic construct
  REGION 1..18: note = Description of sequence: Blood brain barrier penetrating sequence
NSELINSILGLPKVMNDA

SEQ ID NO: 301 - protein, 8 aa, synthetic construct
  REGION 1..8: note = Description of sequence: Blood brain barrier penetrating sequence
EGDFTPRL

SEQ ID NO: 302 - protein, 32 aa, synthetic construct
  REGION 1..32: note = Description of sequence: Blood brain barrier penetrating sequence
EEDRKCPKILMRCKRDSDCLAKCTCQESGYCG

SEQ ID NO: 303 - protein, 9 aa, synthetic construct
  REGION 1..9: note = Description of sequence: Blood brain barrier penetrating sequence
HTAGFIPRL

SEQ ID NO: 304 - protein, 10 aa, synthetic construct
  REGION 1..10: note = Description of sequence: Blood brain barrier penetrating sequence
QLTFTPGWGY

SEQ ID NO: 305 - protein, 13 aa, synthetic construct
  REGION 1..13: note = Description of sequence: Blood brain barrier penetrating sequence
FQFVNPSDIVFGS

SEQ ID NO: 306 - protein, 25 aa, synthetic construct
  REGION 1..25: note = Description of sequence: Blood brain barrier penetrating sequence
GLMSSIGKALGGLIVDVLKPKTPAS

SEQ ID NO: 307 - protein, 9 aa, synthetic construct
  REGION 1..9: note = Description of sequence: Blood brain barrier penetrating sequence
TPSQDFMRF

SEQ ID NO: 308 - protein, 16 aa, synthetic construct
  REGION 1..16: note = Description of sequence: Blood brain barrier penetrating sequence
SSGGGEGSGMWFGPRL

SEQ ID NO: 309 - protein, 39 aa, synthetic construct
  REGION 1..39: note = Description of sequence: Blood brain barrier penetrating sequence
APRLPQCQGDDQEKCLCNKDECPPGQCRFPRGDADPYCE

SEQ ID NO: 310 - protein, 29 aa, synthetic construct
  REGION 1..29: note = Description of sequence: Blood brain barrier penetrating sequence
RICPRILMECKADSDCLAQCICQESGFCG

SEQ ID NO: 311 - protein, 28 aa, synthetic construct
  REGION 1..28: note = Description of sequence: Blood brain barrier penetrating sequence
HSDAVFTDNYSRIRKQMAVKKYINSLLA

SEQ ID NO: 312 - protein, 17 aa, synthetic construct
  REGION 1..17: note = Description of sequence: Blood brain barrier penetrating sequence
GVLDAFKKIATVVKNLV

SEQ ID NO: 313 - protein, 30 aa, synthetic construct
  REGION 1..30: note = Description of sequence: Blood brain barrier penetrating sequence
SIPCGESCVWIPCTITALAGCKCKSKVCYN

SEQ ID NO: 314 - protein, 36 aa, synthetic construct
  REGION 1..36: note = Description of sequence: Blood brain barrier penetrating sequence
YPAKPEAPGEDASPEELSRYYASLRHYLNLVTRQRY

SEQ ID NO: 315 - protein, 9 aa, synthetic construct
  REGION 1..9: note = Description of sequence: Blood brain barrier penetrating sequence
GRAENFLRL

SEQ ID NO: 316 - protein, 7 aa, synthetic construct
  REGION 1..7: note = Description of sequence: Blood brain barrier penetrating sequence
QPWLPFR

SEQ ID NO: 317 - protein, 19 aa, synthetic construct
  REGION 1..19: note = Description of sequence: Blood brain barrier penetrating sequence
FLSLIPHAINAVSALVHHF

SEQ ID NO: 318 - protein, 9 aa, synthetic construct
  REGION 1..9: note = Description of sequence: Blood brain barrier penetrating sequence
CYIQNCPLG

SEQ ID NO: 319 - protein, 9 aa, synthetic construct
  REGION 1..9: note = Description of sequence: Blood brain barrier penetrating sequence
DGGRNFLRF

SEQ ID NO: 320 - protein, 11 aa, synthetic construct
  REGION 1..11: note = Description of sequence: Blood brain barrier penetrating sequence
GRGASSNYVRL

SEQ ID NO: 321 - protein, 10 aa, synthetic construct
  REGION 1..10: note = Description of sequence: Blood brain barrier penetrating sequence
KTCENLADTY

SEQ ID NO: 322 - protein, 37 aa, synthetic construct
  REGION 1..37: note = Description of sequence: Blood brain barrier penetrating sequence
SPTISITAPIDVLRKTWEQERARKQMVKNREFLNSLN

SEQ ID NO: 323 - protein, 9 aa, synthetic construct
  REGION 1..9: note = Description of sequence: Blood brain barrier penetrating sequence
GVDSSFLRL

SEQ ID NO: 324 - protein, 30 aa, synthetic construct
  REGION 1..30: note = Description of sequence: Blood brain barrier penetrating sequence
GLFKTLIKGAGKMLGHVAKEFLGSQGQPES

SEQ ID NO: 325 - protein, 7 aa, synthetic construct
  REGION 1..7: note = Description of sequence: Blood brain barrier penetrating sequence
AQSFVRL

SEQ ID NO: 326 - protein, 6 aa, synthetic construct
  REGION 1..6: note = Description of sequence: Blood brain barrier penetrating sequence
QVHHQK

SEQ ID NO: 327 - protein, 22 aa, synthetic construct
  REGION 1..22: note = Description of sequence: Blood brain barrier penetrating sequence
FLGSIIGALAKGLPSLIALIKK

SEQ ID NO: 328 - protein, 9 aa, synthetic construct
  REGION 1..9: note = Description of sequence: Blood brain barrier penetrating sequence
TPNRDFMRF

SEQ ID NO: 329 - protein, 5 aa, synthetic construct
  REGION 1..5: note = Description of sequence: Blood brain barrier penetrating sequence
IEFFA

SEQ ID NO: 330 - protein, 15 aa, synthetic construct
  REGION 1..15: note = Description of sequence: Blood brain barrier penetrating sequence
GLLDVVGNLLGGLGL

SEQ ID NO: 331 - protein, 12 aa, synthetic construct
  REGION 1..12: note = Description of sequence: Blood brain barrier penetrating sequence
RICTPIPFPMCY

SEQ ID NO: 332 - protein, 13 aa, synthetic construct
  REGION 1..13: note = Description of sequence: Blood brain barrier penetrating sequence
QEKPYWPPPIYPM

SEQ ID NO: 333 - protein, 16 aa, synthetic construct
  REGION 1..16: note = Description of sequence: Blood brain barrier penetrating sequence
TNVKESEFIAEGAAGR

SEQ ID NO: 334 - protein, 36 aa, synthetic construct
  REGION 1..36: note = Description of sequence: Blood brain barrier penetrating sequence
GPSQPTYPGDDAPVEDLIRFYNDLQQYLNVVTRHRY

SEQ ID NO: 335 - protein, 6 aa, synthetic construct
  REGION 1..6: note = Description of sequence: Blood brain barrier penetrating sequence
IAYKPE

SEQ ID NO: 336 - protein, 12 aa, synthetic construct
  REGION 1..12: note = Description of sequence: Blood brain barrier penetrating sequence
DHLPHDVYSPRL

SEQ ID NO: 337 - protein, 13 aa, synthetic construct
  REGION 1..13: note = Description of sequence: Blood brain barrier penetrating sequence
VLPLISMALGKLL

SEQ ID NO: 338 - protein, 7 aa, synthetic construct
  REGION 1..7: note = Description of sequence: Blood brain barrier penetrating sequence
EPYAFGL

SEQ ID NO: 339 - protein, 9 aa, synthetic construct
  REGION 1..9: note = Description of sequence: Blood brain barrier penetrating sequence
CYISNCPIG

SEQ ID NO: 340 - protein, 4 aa, synthetic construct
  REGION 1..4: note = Description of sequence: Linker
GGGS

SEQ ID NO: 341 - protein, 8 aa, synthetic construct
  REGION 1..8: note = Description of sequence: Linker
  REPEAT 5..8: note = Subunit GGGS repeated 2x
GGGSGGGS

SEQ ID NO: 342 - protein, 16 aa, synthetic construct
  REGION 1..16: note = Description of sequence: Linker
  REPEAT 5..16: note = Subunit GGGS repeated 4x
GGGSGGGSGGGSGGGS

SEQ ID NO: 343 - protein, 32 aa, synthetic construct
  REGION 1..32: note = Description of sequence: Linker
  REPEAT 5..32: note = Subunit GGGS repeated 8x
GGGSGGGSGGGSGGGSGGGSGGGSGGGSGGGS